(19) United States
(12) Patent Application Publication
(10) Pub. No.: US 2017/0108482 A1
(43) Pub. Date: Apr. 20, 2017

(54) ANALYSIS DEVICE FOR ANALYZING WATER AND WASTEWATER

(71) Applicant: LAR PROCESS ANALYSERS AG, Berlin (DE)

(72) Inventors: Werner Arts, Berlin (DE); Wolfgang Genthe, Berlin (DE)

(73) Assignee: LAR PROCESS ANALYSERS AG, Berlin (DE)

(21) Appl. No.: 15/314,271

(22) PCT Filed: May 22, 2015
(86) PCT No.: PCT/DE2015/200321

§ 371 Date: Nov. 28, 2016

(30) Foreign Application Priority Data
May 26, 2014 (DE) 202014102443.3

(51) Int. Cl. G01N 33/18; G01N 25/00

(52) U.S. Cl. CPC G01N 33/1806; G01N 25/00

(57) ABSTRACT

Disclosed is an analysis device, in particular for analyzing water and wastewater, comprising a device housing that holds device components and has an opening on a housing surface, said opening being openable and closeable without using any tool; and comprising a component carriage or component insert which supports some of the device components and is configured and arranged together with said device components in such a way that at least part of the carriage or insert can be conveyed out of the housing through the opening so that the device components placed thereon are easily accessible.

### BACKGROUND

The invention relates to an analysis device, in particular for analyzing water and wastewater, having a device housing that holds device components.

Such analysis devices are typically provided with closed housings which need to be partially disassembled for maintenance operations or testing or setting processes that do not take place very frequently. For devices requiring relatively frequent testing, setting or maintenance of certain components, the housing is sometimes realized like a cabinet, thus provided with a door occupying the front which can also be realized as a glass door for the purpose of a first visual inspection of the device components.

### SUMMARY

The invention is based on the task of proposing an analysis device of the generic kind which is improved with respect to its practical handling.

This task is solved by a first aspect of the invention by an analysis device with one or more features of the invention. Appropriate further developments of the inventive concept are the subject matter of the dependent claims.

The invention firstly includes the concept of providing an opening on a housing surface which is openable and closable without using any tool and configured such that device components in need of testing or maintenance can be brought out of the housing through the opening. According to a second concept, a component carriage or component insert is provided for this purpose which supports the relevant device components and is configured and arranged together with them in such a way that it can be conveyed at least partially to the outside. A carriage is in this case understood to be a member that is displaceable in a sliding guide, whereas an insert is understood to be a member that can be conveyed through the opening in another way (such as by manually lifting out or else by pivoting).

According to a first embodiment of the invention, a pivot door or flap hinged to a housing edge is provided for closing the opening. In contrast to known analysis devices, however, such a door is not configured as a member that occupies the entirety of the housing front. In an alternative embodiment, a closing plate is provided at the front edge of the component carriage or component insert for closing the opening when the component carriage is retracted or the component insert is inserted.

In one further configuration of both of the embodiments, the pivot door or flap or closing plate is perforated for enabling air to be exchanged and/or for dissipating heat from the housing's interior.

In further configurations of the invention, the opening in the device housing occupies only a part of the housing front, in particular between 25 and 75%, and more specifically between 40 and 60%, and the component carriage or component insert configured to be adapted thereto occupies a corresponding proportion of the device housing's volume. This ensures that operating and/or display elements can be mounted to the remaining part of the housing front. In an alternative configuration, the opening in the device housings is provided in a lateral surface thereof such that the housing front is essentially completely usable for operating and/or display elements.

According to a relatively independent second aspect of the invention, an analysis device with additional features is moreover proposed. In same, the device housing that holds device components has an input opening on a housing surface for inputting a substance to be analyzed into a device component when the device housing is closed.

In a first embodiment of the device according to that aspect, the input opening is formed as an injection port for injecting a liquid. In an alternative configuration, the input opening is formed as a closable, in particular self-closing media chute for inputting a solid or free flowing sample.

According to a further embodiment, the input opening is disposed on the upper side of the device and in particular above the component carriage or component insert which is provided according to the aspects of the invention described further above. Specifically, the input opening, that is in particular formed as an injection port, is in communication with a reaction vessel for thermally decomposing the substance to be analyzed.

### BRIEF DESCRIPTION OF THE DRAWINGS

Advantages and utilities of the invention will incidentally result from the following brief description of an exemplary embodiment with reference to the figures. Shown are in:

FIG. 1 a schematic representation of essential device components of an analysis device according to an exemplary embodiment, and

FIG. 2 a perspective representation of that analysis device.

### DETAILED DESCRIPTION OF THE PREFERRED EMBODIMENTS

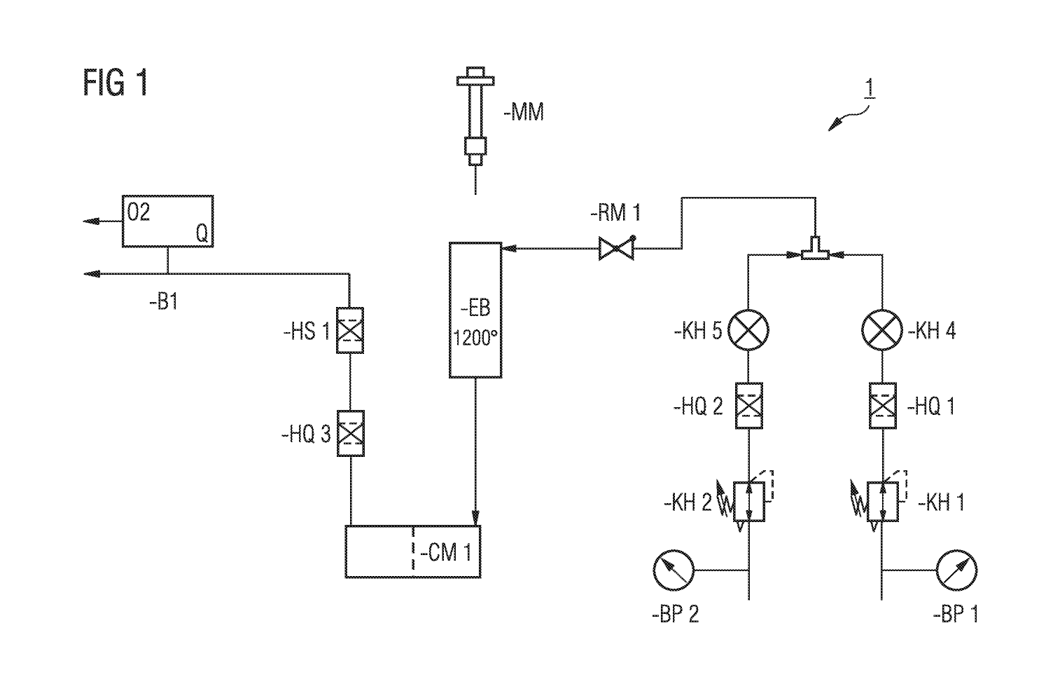

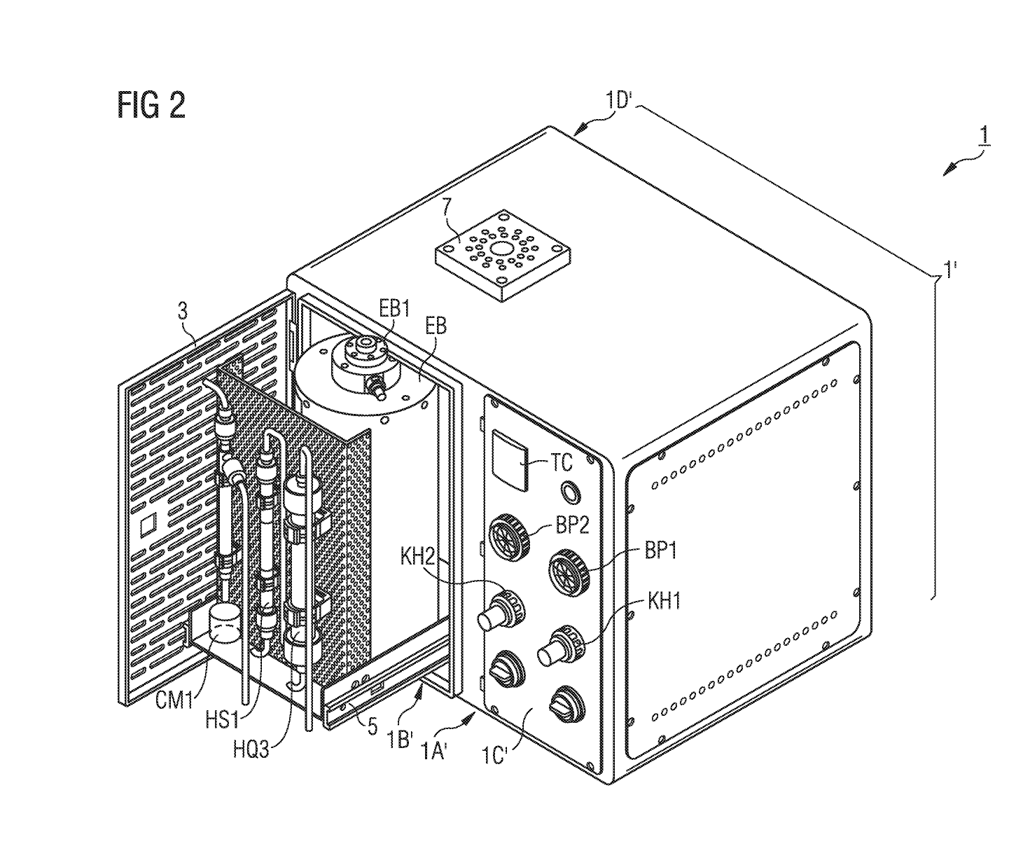

FIGS. 1 and 2 schematically show essential parts of an analysis device 1 for determining the chemical oxygen demand (COD) of water or wastewater. The fundamental structure of such devices and the functionality thereof are known to the skilled person and will thus not be described here in more detail, particularly since it is not of relevance for understanding the present invention.

The analysis device 1 comprises a thermal reaction vessel and a furnace EB respectively, in which a water sample can be injected by means of an injection gun MM, and in which this sample is thermally decomposed. A carrier gas flow composed of air and nitrogen is supplied to the furnace via a check valve RM1. The carrier gas flow is controlled by means of an air pressure governor KH1, a nitrogen pressure governor KH2, an air flow rate governor KH4 and a nitrogen flow rate governor KH5, and is filtered by means of a first and second fine filter HQ1, HQ2 at the input side of the furnace. At the input side of the furnace, an air pressure display BP1 and a nitrogen pressure display BP2 are also provided.

At the output side of the furnace the gas flow firstly moves into a condensate container CM1, and the non-condensed fraction subsequently flows through a quartz wool filter HQ3 and an acid trap HS1 before reaching the oxygen detector B1 which finally outputs an (electric) measured value to further device components (not shown) for signal processing, storage and representation.

FIG. 2 shows the analysis device 1 in a perspective view in a state in which the device housing 1′ is partially opened and part of the device components have been pulled out of the housing. The device housing essentially has the shape of a square prism, and in the housing front 1A′, an opening 1B′ is provided which can be closed by a perforated door 3 hinged at the left-hand lateral edge of the housing front.

A carrier 5 dimensionally adapted to the opening 1B′, on which the furnace EB, the condensate container CM1, the quartz wool filter HQ3 and the acid trap HS1 are disposed, may be extracted from the housing thus far that the mentioned components become easily accessible. In the carriage's 5 pushed back state, the device housing 1′ will be closed by the door 3.

On the right-hand side of the device front 1A′, a control panel 1C′ is situated on which several operating and display elements are arranged, including a temperature display/control TC and the setting governors KH1 and KH2 for the air pressure or nitrogen pressure, and the associated display elements BP1 and BP2.

On the upper side 1D′ of the device, an injection port 7 is situated, the structure and dimensions of which are adapted to those of the injection gun MM shown in FIG. 1, and which communicates with a corresponding injection valve EB 1 of the furnace EB in the device's interior, when the furnace is in its normal position of use inside the device housing.

The configuration of the invention is not restricted to this example but is likewise possible in a multitude of modifications which are within the skilled person's scope.

1. An analysis device, comprising
a device housing that holds device components and has an opening on a housing surface, said opening being openable and closable without using any tools, and
a component carriage or component insert which supports part of the device components and is configured and arranged together with said device components such that at least part of the carriage or insert is conveyable out through the opening so that the device components placed thereon are easily accessible.

2. The analysis device according to claim 1, further comprising a pivot door or flap hinged to a housing edge that closes the opening.

3. The analysis device according to claim 1, further comprising a closing plate at a front edge of the component carriage or component insert for closing the opening when the component carriage is retracted or the component insert is inserted.

4. The analysis device according to claim 2, wherein the pivot door or flap is perforated for enabling at least one of air to be exchanged or for dissipating heat from an interior of the housing.

5. The analysis device according to claim 1, wherein the opening in the device housing occupies only a part of a housing front, and the component carriage or component insert configured to be adapted thereto occupies a corresponding proportion of the device housing's volume, at least one of operating or display elements being provided on a remaining part of the housing front.

6. The analysis device according to claim 1, wherein the opening in the device housing is provided in a lateral surface thereof such that a housing front is essentially completely usable for at least one of operating or display elements.

7. An analysis device according to claim 1, further comprising
an input opening on the housing surface for inputting a substance to be analyzed into a device component when the device housing is closed.

8. The analysis device according to claim 7, wherein the input opening is formed as an injection port for injecting a liquid.

9. The analysis device according to claim 7, wherein the input opening is formed as a closable media chute for inputting a solid or free flowing sample.

10. The analysis device according to claim 7, wherein the input opening is disposed on an upper side of the device.

11. The analysis device according to claim 7, wherein the input opening is in communication with a reaction vessel for thermally decomposing a substance to be analyzed.

12. The analysis device of claim 1, wherein the analysis device is for analyzing water or wastewater.

13. The analysis device according to claim 3, wherein the closing plate is perforated for enabling at least one of air to be exchanged or for dissipating heat from an inside of the housing.

14. The analysis device of claim 5, wherein the opening in the device housing occupies 25% to 75% of the housing front.

15. The analysis device according to claim 10, wherein the input opening is above the component carriage or component insert.
